(19) United States
(12) Patent Application Publication
(10) Pub. No.: US 2003/0104603 A1
(43) Pub. Date: Jun. 5, 2003

(54) Protein

(76) Inventors: Peter Lind, Uppsala (SE); Charlotte Soderberg, Uppsala (SE)

Correspondence Address: JANIS K. FRASER, PH.D., FISH & RICHARDSON P.C., 225 Franklin Street, Boston, MA, 02110-2804, US

(21) Appl. No.: 10/187,171

(22) Filed: Jun. 27, 2002

Related U.S. Application Data
(60) Provisional application No. 60/300,814, filed on Jun. 27, 2001.

(30) Foreign Application Priority Data
Jun. 27, 2001 (SE) 0102290-4

(51) Int. Cl. C12N 9/88; C07H 21/04; C12P 21/02; C12N 5/06

(52) U.S. Cl. 435/232; 435/69.1; 435/320.1; 435/325; 536/23.2

(57) ABSTRACT

The present invention relates to citrate lyase polypeptides and to nucleic acid molecules coding for such polypeptides. Citrate lyase is transcriptionally regulated in adipose tissue, suggesting involvement in a novel pathway in energy expenditure, downstream of fatty acid metabolism. This makes CitE an attractive drug target for treating and/or preventing diseases such as obesity. Accordingly, the invention refers to the novel mammalian CitE-polypeptide, as well as homologs and functionally equivalent variants thereof. Further, the invention refers to the nucleic acid sequence encoding the novel protein and to vectors for expressing the protein in various organisms. The invention also refers to methods for expressing the protein in said organisms and for purifying the protein upon overexpression. Moreover, the invention refers to a methods wherein CitE is used for screening for substances that affect the activity of CitE.

### CROSS REFERENCE TO RELATED APPLICATIONS

This application claims priority from Swedish Patent Application No. 0102290-4, filed Jun. 27, 2001, and U.S. Provisional Patent Application Serial No. 60/300,814, filed Jun. 27, 2001. These applications are incorporated herein by reference in their entirety.

### TECHNICAL FIELD

The invention relates to isolated nucleic acids encoding mammalian CitE polypeptides, as well as mammalian CitE polypeptides. Furthermore, the invention relates to methods for screening for potential drug targets related to fatty acid metabolism.

### TECHNICAL BACKGROUND

Obesity is a chronic, metabolic disease caused by multiple and complex inherited and acquired factors, including excessive calorie and food intake, decreased physical activity, and genetic influences. The defining characteristic is excess body fat. Long-term treatment and management are required to achieve and sustain weight loss. Today, more than 70 million Americans are overweight. Obesity, particularly when associated with unhealthy patterns of body fat distribution, results (directly or indirectly) in 300,000 preventable deaths each year in the U.S. and $100 billion in health care costs. In the last ten years, the obese proportion of the population has increased from 25 percent to 32 percent—a level that may be considered epidemic. The same pattern, though perhaps less pronounced, is also seen in the rest of the western world, including Japan.

A number of conditions are tightly associated with obesity. These conditions include; cardiovascular disease, high blood pressure, type 2 diabetes, elevated blood cholesterol and triglyceride levels, gout, sleep apnea/obesity hyperventilation syndrome, osteoarthritis of the weight bearing joints and infertility. Obesity also increases the risk for developing certain forms of cancer (e.g., breast and colorectal cancer).

An excess of body fat results from an imbalance between energy intake and energy output (i.e., consuming more calories than are needed to support your body's energy needs). The reasons for this imbalance are unclear, and the relationship between energy intake/expenditure and body fat storage and distribution varies from person to person. Factors that promote obesity include a genetic predisposition, family history of obesity, age behavioral factors (such as a high fat diet and sedentary lifestyle), and biochemical differences (lower metabolic rate or decreased ability to oxidize fat).

Most drugs currently on the market for obesity influence the levels of serotonin or of noradrenaline in specific areas of the brain that regulate food intake, energy expenditure and body weight. These drugs can be classified as either serotonergic or adrenergic. Both types control the appetite by reducing hunger and/or increasing satiety. They may also increase total daily energy expenditure.

Adrenergic drugs include Phentermine (Adipex, Fastin, Ionamin), diethylpropion (Tenuate, Tepanil), mazindol (Sanorex, Mazinor), phendimetrazine (Adipost, Bontril, Plegine, Prelu-2), and benzphetamine (Didrex). Apart from the side effects that include dry mouth, anxiety, insomnia, dizziness, palpitations and (rarely) increased blood pressure, these drugs also suffer from the drawback that they are all classified as controlled substances by the U.S. Drug Enforcement Agency (DEA). These drugs are approved by the U.S. Food and Drug Administration (FDA) for short-term (about 12 weeks) use only. Orlistat (Xenical) does not act directly on the central nervous system. It inhibits an enzyme (pancreatic lipase) essential to fat digestion. The most common side effects of orlistat are intestinal symptoms, including cramping, gas and diarrhea—particularly in patients who ate high-fat foods. These side effects are not pleasant and it is possible that the desire to avoid these side effects encourages people to eat a diet that is lower in fat, thereby helping them to lose weight.

Despite the many available drugs against obesity, it is clear that none of them are efficient and/or without complications in the treatment of obesity.

The complexity of the disease makes it difficult to find new drug targets towards which new drug can be developed. There exists an urgent need to discover novel drug targets for the treatment and/or prevention of the above mentioned diseases.

### SUMMARY OF THE INVENTION

The inventors of the present invention have surprisingly found a mammalian ortholog to the bacterial CitE. A similar enzyme is present in bacteria, but it has never been described in humans. This enzyme is transcriptionally regulated in adipose tissue. The enzyme is involved in a novel pathway in energy expenditure, downstream of fatty acid metabolism. The suggestion that down-regulation of CitE leads to lipolysis and energy expenditure, makes CitE an attractive drug target for treating and/or preventing diseases such as those mentioned above. The fact that CitE is a cytosolic enzyme that is to be inhibited, makes it an even more attractive drug target.

Accordingly, the invention refers to the novel mammalian CitE-polypeptide, as well as homologs and functionally equivalent variants thereof. Further, the invention refers to the nucleic acid sequence encoding the novel protein and to vectors for expressing the protein in various organisms. The invention also refers to methods for expressing the protein in said organisms and for purifying the protein upon overexpression. Moreover, the invention refers to a novel method wherein CitE is used for screening for substances that affect the activity of CitE.

One further potential application of the human sequence is to use it as a screening target for potential drug molecules. The rat sequence may be used as a target for testing the expected efficiency of various substances (which have been screened against a human receptor) in an animal model. This might be relevant, since it is not at forehand clear that a substance working as an inhibitor or enhancer towards the human enzyme also has the same effect on the rat enzyme.

### BRIEF DESCRIPTION OF THE DRAWINGS

FIG. 1 is a photograph of a Northern blot analysis showing the expression pattern of the CitE homolog in mouse and rat tissues, respectively.

FIG. 2 is a graph depicting CitE expression as detected in a human Multiple Tissue Expression array (MTE).

FIG. 3 is a graph depicting CitE expression as detected in a human Multiple Tissue Northern (MTN).

### DETAILED DESCRIPTION OF THE INVENTION

The inventors of the present invention have shown that the stimulation of lipolysis and energy expenditure in mice by the specific β3-agonist CL 316,243 induces transcriptional changes in many genes in mouse adipose tissue, as detected by the Affymetrix GeneChip technology. One of these genes, represented by ESTs only, and which was down-regulated, encodes a protein with apparent homology to bacterial citrate lyase beta chain (CitE), as well as to the Caenorhabditis elegans gene product C01G10.7. The bacterial enzyme cleaves citrate to acetyl-moieties and oxaloacetate, and has a structure and molecular mechanism that is distinct from the ATP-dependent citrate lyase.

The metabolism of citrate is a central pathway in fatty acid metabolism. In mammalians, citrate is normally transformed into acetyl-units and oxaloacetate by an ATP-dependent citrate lyase. In bacteria, a different class of citrate lyases (CitE) is known, performing the same reaction, but in an ATP-independent manner. Variants of this bacterial enzyme are described in, for example, Bekal et al. (J. Bacteriology, February 1998; 180(3): 647-654), which discloses the purification of Leuconostoc mesenteroides citrate lyase and the cloning and characterization of the corresponding gene cluster. Furthermore, Bott (Arch Microbiol (1997) 167:78-88) discloses anaerobic citrate metabolism and its regulation in enterobacteria, and Arps et al. (J Bacteriol, June 1993; 175(2): 3776-83) discloses the genetics of malyl coenzyme A lyase in Methylobacterium extorquens AM1.

Using Rapid amplification of cDNA Ends (RACE) PCR the inventors were able to clone the mouse and rat homologs to bacterial CitE. Using the sequences of these clones, the inventors found a human ortholog to CitE, which has not previously been described.

The term “CitE” refers to the enzyme citryl-CoA lyase.

The term “hCitE” refers to the human homolog of CitE.

The hCitE protein consists of 340 amino acids and is shown in (SEQ ID NO: 2).

The amino acid identity between the mammalian and bacterial enzymes range between 27% and 30%. This level of identity suggests that the proteins share the same three-dimensional structure.

In one aspect, the invention features an isolated nucleic acid molecule containing a nucleotide sequence that is at least 60% identical to the nucleotide sequence shown in SEQ ID NO: 1, SEQ ID NO: 3, or SEQ ID NO: 5. More preferably, the nucleotide sequence is at least 65%, 70%, 75%, 80%, 85%, 90%, 91%, 92%, 93%, 94%, 95%, 96%, 97%, 98%, 99%, or more, identical to the nucleotide sequence shown in SEQ ID NO: 1, SEQ ID NO: 3, or SEQ ID NO: 5. In the case of a nucleotide sequence that is longer than or equivalent in length to the reference sequence, e.g., SEQ ID NO: 1, SEQ ID NO: 3, or SEQ ID NO: 5, the comparison is made with the full length of the reference sequence. Where the nucleotide sequence is shorter that the reference sequence, e.g., shorter than SEQ ID NO: 1, SEQ ID NO: 3, or SEQ ID NO: 5, the comparison is made to a segment of the reference sequence of the same length (excluding any loop required by the homology calculation). Preferably, the nucleotide sequence encodes a polypeptide having citrate lyase activity. In one example, the nucleotide sequence is identical to SEQ ID NO: 1, SEQ ID NO: 3, or SEQ ID NO: 5.

In one embodiment, the invention features an isolated nucleic acid comprising a nucleotide sequence that encodes a polypeptide comprising an amino acid sequence that is at least about 60% identical to a sequence shown as SEQ ID NO: 2, SEQ ID NO: 4, SEQ ID NO: 6, or a fragment thereof. Preferably, the amino acid sequence is at least 65%, 70%, 75%, 80%, 85%, 90%, 95%, 98%, 99% or more identical to SEQ ID NO: 2, SEQ ID NO: 4, or SEQ ID NO: 6 and has a citrate lyase activity described herein. For example, the amino acid sequence can be identical to the sequence of SEQ ID NO: 2, SEQ ID NO: 4, or SEQ ID NO: 6.

A sequential grouping of three nucleotides, a “codon,” codes for one amino acid. Since there are 64 possible codons, but only 20 natural amino acids, most amino acids are coded for by more than one codon. This natural “degeneracy”, or “redundancy”, of the genetic code is well known in the art. It will thus be appreciated that the nucleotide sequences of SEQ ID NO: 1, SEQ ID NO: 3, and SEQ ID NO: 5 are only examples within a large but definite group of DNA sequences that encode the polypeptides described herein.

Also included in the invention is an isolated nucleic acid molecule comprising a nucleotide sequence that hybridizes under stringent hybridization conditions to the nucleotide sequence of SEQ ID NO: 1, SEQ ID NO: 3, and SEQ ID NO: 5, the complete complement of SEQ ID NO: 1, SEQ ID NO: 3, and SEQ ID NO: 5, or a segment thereof as described herein. The term “stringent hybridization conditions” is known in the art from standard protocols (e.g., Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989) and could be understood as e.g., hybridization to filter-bound DNA in 0.5 M NaHPO₄, 7% sodium dodecyl sulfate (SDS), 1 mM EDTA at +65° C., and washing in 0.1×SSC/0.1% SDS at +68° C. Preferably, the nucleotide sequence encodes a polypeptide having citrate lyase activity.

Also included in the invention is an isolated nucleic acid molecule comprising a nucleotide sequence that hybridizes under high stringency conditions to the nucleotide sequence of SEQ ID NO: 1, SEQ ID NO: 3, or SEQ ID NO: 5, the complete complement of SEQ ID NO: 1, SEQ ID NO: 3, or SEQ ID NO: 5, or a segment thereof as described herein. “High stringency conditions” refers to hybridization in 6×SSC at about 45° C., followed by one or more washes in 0.2×SSC, 0.1% SDS at 65° C. Preferably, the nucleotide sequence encodes a polypeptide having citrate lyase activity.

Also included in the invention is an isolated nucleic acid molecule comprising a nucleotide sequence that encodes a polypeptide comprising a functional domain of the polypeptide of SEQ ID NO: 2, SEQ ID NO: 4, or SEQ ID NO: 6 described herein, e.g., a citrate lyase domain of a citrate lyase of SEQ ID NO: 2, SEQ ID NO: 4, or SEQ ID NO: 6. Preferably, the nucleotide sequence encodes a polypeptide having citrate lyase activity.

Also included in the invention is an isolated nucleic acid molecule comprising a nucleotide sequence that encodes a polypeptide comprising at least 20, 30, 40, 50, 75, 100, 150, 200, 250, 300, 325, or more contiguous amino acid residues of SEQ ID NO: 2, SEQ ID NO: 4, or SEQ ID NO: 6. In some embodiments, the polypeptide comprises an immunogenic fragment of at least 20 amino acids of SEQ ID NO: 2, SEQ ID NO: 4, or SEQ ID NO: 6. Preferably, the nucleotide sequence encodes a polypeptide having citrate lyase activity.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

As used herein, “% identity” of two amino acid sequences or of two nucleic acid sequences is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

In another aspect, the invention features a substantially pure polypeptide having a sequence shown as SEQ ID NO: 2, SEQ ID NO: 4, or SEQ ID NO: 6. The invention also includes a polypeptide, or fragment thereof, that differs from the corresponding sequence shown as SEQ ID NO: 2, SEQ ID NO: 4, or SEQ ID NO: 6. The differences are, preferably, differences or changes at a non-essential residue or a conservative substitution. In one embodiment, the polypeptide includes an amino acid sequence at least about 60% identical to a sequence shown as SEQ ID NO: 2, SEQ ID NO: 4, or SEQ ID NO: 6, or a fragment thereof. Preferably, the amino acid sequence is at least 65%, 70%, 75%, 80%, 85%, 90%, 95%, 98%, 99% or more identical to SEQ ID NO: 2, SEQ ID NO: 4, or SEQ ID NO: 6 and has a citrate lyase activity described herein. For example, the amino acid sequence can be identical to SEQ ID NO: 2, SEQ ID NO: 4, or SEQ ID NO: 6.

Preferred polypeptide fragments of the invention are at least 10%, preferably at least 20%, 30%, 40%, 50%, 60%, 70%, or more, of the length of the sequence shown as SEQ ID NO: 2, SEQ ID NO: 4, or SEQ ID NO: 6 and have a citrate lyase activity described herein. Alternatively, the fragment can be merely an immunogenic fragment, e.g., a fragment that can be used to raise monoclonal and/or polyclonal antibodies that specifically bind to a polypeptide of the sequence of SEQ ID NO: 2, SEQ ID NO: 4, or SEQ ID NO: 6.

Included in the invention are variants, derivatives, and fragments of a mammalian CitE polypeptide. The skilled person will readily be able to determine whether such a variant, derivative, or fragment of a mammalian CitE polypeptide displays CitE activity by subjecting the variant, derivative, or fragment to a standard citrate lyase assay. Such assays are well known in the art (see e.g. Bekal S, et al. Purification of Leuconostoc mesenteroides citrate lyase and cloning and characterization of the citCDEFG gene cluster.J Bacteriol. February 1998; 180(3):647-54.).

The invention encompasses polypeptides carrying modifications such as substitutions, small deletions, insertions or inversions, which polypeptides nevertheless have substantially the biological activities of a citrate lyase. Also included in the invention is a polypeptide encoded by a nucleic acid molecule described herein.

By a “functionally equivalent form” is meant a form of the protein, which possesses essentially the same activity, e.g., a citrate lyase activity, as a full length citrate lyase polypeptide. A functionally equivalent form preferably comprises at least 100 amino acids, more preferably at least 200 amino acids, most preferably at least 300 amino acids.

Also included in the invention is a substantially pure polypeptide comprising a functional domain of the polypeptide of SEQ ID NO: 2, SEQ ID NO: 4, or SEQ ID NO: 6 described herein, e.g., a catalytic domain. Preferably, the polypeptide has citrate lyase activity.

Also included in the invention is a substantially pure polypeptide comprising at least 20, 30, 40, 50, 75, 100, 150, 200, 250, 300, 325, or more contiguous amino acid residues of SEQ ID NO: 2, SEQ ID NO: 4, or SEQ ID NO: 6. In some embodiments, the polypeptide comprises an immunogenic fragment of at least 20 amino acids of SEQ ID NO: 2, SEQ ID NO: 4, or SEQ ID NO: 6. Preferably, the polypeptide has citrate lyase activity.

The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis.

The present invention also relates to vectors comprising the nucleic acid molecules of the invention, as well as host cells transformed with such vectors. Any of the nucleic acid molecules of the invention may be joined to a vector, which generally includes a selectable marker and an origin of replication for propagation in a host cell. Because the invention also provides polypeptides expressed from the nucleic acid molecules described above, vectors for the expression of a polypeptide are preferred. Such expression vectors include DNA encoding a polypeptide described herein, operably linked to suitable transcriptional or translational regulatory sequences, such as those derived from a mammalian, microbial, viral, or insect gene. Examples of regulatory sequences include transcriptional promoters, operators, or enhancers, mRNA ribosomal binding sites, and appropriate sequences, which control transcription and translation. Nucleotide sequences are operably linked when the regulatory sequence functionally relates to the DNA encoding a polypeptide. Thus, for example, a promoter nucleotide sequence is operably linked to a Cit E DNA sequence if the promoter nucleotide sequence directs the transcription of the Cit E sequence.

The vector may be any vector, which conveniently may be subjected to recombinant DNA procedures, and the choice of vector will often depend on the host cell into which it is to be introduced. Thus, the vector may be an autonomously replicating vector, e.g., a vector that exists as an extrachromosomal entity, the replication of which is independent of chromosomal replication (examples of such a vector are a plasmid, phage, cosmid, mini-chromosome or virus). Alternatively, the vector may be one which, when introduced in a host cell, is integrated in the host cell genome and replicated together with the chromosome(s) into which it has been integrated. Examples of suitable vectors are a bacterial expression vector and a yeast expression vector. The vector of the invention may carry any of the DNA molecules of the invention as defined above.

A suitable host cell can be a prokaryotic cell, a unicellular eukaryotic cell, or a cell derived from a multicellular organism. The host cell can thus, e.g., be a bacterial cell such as an E. coli cell, a cell from a yeast such as Saccharomyces cervisiae or Pichia pastoris, an insect cell, or a mammalian cell, such as HEK293, CHO or an equivalent. The methods employed to effect introduction of the vector into the host cell are standard methods well known to a person familiar with recombinant DNA methods.

Included in the invention is a process for production of a polypeptide described herein, which comprises culturing a host cell as described above under conditions whereby said polypeptide is produced, and optionally recovering said polypeptide, using standard biochemical procedures.

A further aspect of the invention is a nucleic acid probe comprising at least 15 nucleotides, which probe specifically hybridizes with at least a part of the nucleic acid molecule according to the invention, said part having a sequence shown in SEQ ID NO: 1, SEQ ID NO: 3, or SEQ ID NO: 5. The invention also provides an antisense oligonucleotide having a sequence capable of specifically hybridizing to at least a part of the nucleic acid molecule according to the invention, said part having a sequence shown in SEQ ID NO: 1, SEQ ID NO: 3, or SEQ ID NO: 5.

Fragments of the nucleic acid molecules described herein, as well as polynucleotides capable of hybridizing to such nucleic acid molecules may be used as a probe or as primers in a polymerase chain reaction (PCR). Such probes may be used, e.g., to detect the presence of nucleic acids coding for Cit E in in vitro assays, as well as in Southern and Northern blots. Cell types expressing Cit E may also be identified by the use of such probes. Such procedures are well known, and the skilled artisan will be able to choose a probe of a length suitable to the particular application. For PCR, 5′ and 3′ primers corresponding to the termini of a desired Cit E nucleic acid molecule are employed to isolate and amplify that sequence using conventional techniques.

The polypeptides of the present invention may also be used to raise polyclonal and monoclonal antibodies, which are useful in diagnostic assays for detecting Cit E polypeptide expression. Such antibodies may be prepared by conventional techniques. See, for example, Antibodies: A Laboratory Manual, Harlow and Land (eds.), Cold Spring Harbor Laboratory Press, Cold Spring Harbor, N.Y., (1988); Monoclonal Antibodies, Hybridomas: A New Dimension in Biological Analyses, Kennet et al. (eds.), Plenum Press, New York (1980).

Included in the invention is a method for screening for a compound that modulates (increases or decreases) an activity of a citrate lyase, the method comprising: contacting the a polypeptide described herein with a test compound; measuring a citrate lyase activity of the polypeptide in the presence of the test compound; comparing the citrate lyase activity of the polypeptide in the presence of the test compound with the citrate lyase activity of the polypeptide in the absence of the test compound, to thereby determine whether the test compound modulates an activity of a citrate lyase. The method can be used, for example, to screen for compounds that increase or decrease the effect or activity of citrate lyase, with the potential of alleviating pathogenic effects of defects in citrate metabolism.

As a “test compound” is meant any suitable molecule being a potential drug target compound.

Furthermore, the invention provides a kit for carrying out the screening method above, comprising a polypeptide described herein, reagents for performing the method, and optionally instructions for use.

Included in the invention is a method of identifying an agent that binds to a citrate lyase, the method comprising: contacting a polypeptide comprising the amino acid sequence of SEQ ID NO: 2, SEQ ID NO: 4, or SEQ ID NO: 6 with a candidate agent; and determining that the candidate agent binds to the polypeptide, to thereby identify a candidate agent that binds to a citrate lyase.

The invention also provides a method for disrupting a citrate lyase (Cit E) gene in a non-human embryonic stem cell, the method comprising: providing a nucleotide sequence (e.g., an oligonucleotide) capable of disrupting a Cit E gene; and introducing the nucleotide sequence into a non-human embryonic stem-cell under conditions such that nucleotide sequence is homologously recombined into the Cit E gene in the genome of the cell, to produce a cell containing at least one disrupted Cit E allele. The invention further provides a non-human transgenic animal expressing reduced levels of Cit E, wherein the Cit E gene has been disrupted by the above method.

The invention also provides a non-human transgenic animal whose genome comprises an antisense nucleic acid molecule that hybridizes to an mRNA encoding a polypeptide described herein, thereby reducing translation of the polypeptide in the animal. A transgenic non-human mammal can e.g. be mammalian animal, such as a mouse, or an animal of the species Caenorhabditis elegans.

The invention also provides a method for screening for compounds that affect a fatty acid metabolism pathway, the method comprising: providing a non-human transgenic animal described herein; providing a composition comprising a test compound in a form suitable for administration to the non-human animal; administering the test compound to the non-human animal; and determining the effect of the test compound on a fatty acid metabolism pathway in the animal. The method can be used to identify compounds that either increase or decrease the effect or activity of any molecule downstream of the a fatty acid metabolism, with the potential of alleviating the pathogenic effects of defects in a fatty acid metabolism.

Still further, the invention refers to the use of a substance identified by a method described herein for use in the treatment of diseases such as diabetes type 2, impaired glucose tolerance and obesity related insulin resistance. Impaired insulin resistance and associated glucose intolerance can occur in type 2 diabetes, type 1 diabetes, maturity-onset diabetes of the young (MODY), gestational diabetes, obesity and diseases related to the metabolic syndrome such dyslipedemia, atherosclerosis hypertension. Impaired insulin resistance and glucose intolerance can also occur in non-diabetic individuals and is considered as a predisposing factor for type 2 diabetes. Obesity is a condition in which there is an increase in body fat content resulting in excess body weight above accepted norms for age gender height and body build. An excess of mortality that occurs in obese individuals results from diseases that are predisposed by this condition. They include cancer, cardiovascular disease, digestive disease, respiratory disease and type 2 diabetes. In patients with chronic hyperglycemia such as type 2 diabetes, glucose dependent protein cross-linking occurs at a rate in excess of the norm. Excessive non-enzymatic glycosylation of proteins contributes to diabetic complications and complications of aging in non-diabetic humans such as neuropathy, nephropathy, retinopathy, hypertension and atherosclerosis.

Reducing the insulin resistance and the abnormally high blood glucose level of diabetic subjects benefits the patient by reducing the discomfort of glucosuria and the excessively high mortality and morbidity associated with metabolic syndrome.

The invention also includes a method for the treatment of a disorder selected from the group consisting of type 2 diabetes, type 1 diabetes, MODY, gestational diabetes, obesity, dyslipidemia, hypertension, cancer, cardiovascular disease, digestive disease, respiratory disease, neuropathy, nephropathy, retinopathy, hypertension, and atherosclerosis, the method comprising administering to an individual in need thereof an amount of an agent effective to modulate (increase or decrease) the expression or activity, e.g., a citrate lyase activity, of a Cit E polypeptide. The method can include an additional step of diagnosing an individual as having a disorder described herein.

In yet another aspect, the invention refers to a substrate molecule for the enzyme CitE, which substrate molecule is represented by formula I:

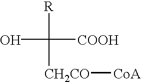

wherein R is CH₂COOH, H, CH₃, CH₃(CH₂)ₙ or (CH₂)ₘCOOH, whereby n is 1, 2, 3, 4, 5 or 6,and m is 2, 3, 4, 5, 6or 7.

In bacteria, some examples of substrates are R═CH₂COOH (citryl-CoA metabolism) and R═H (malyl-CoA-metabolism).

The substrate molecule may be cleaved into acetyl-moieties and oxalo-acetate, in which reaction Cit E functions as a catalyst, as is the case with CitE in bacteria. Malyl-CoA-lyase in bacteria gives the products acetyl-CoA and glyoxalate. Accordingly, the mammalian enzyme is expected to give acetyl-CoA and different alfa-keto-carboxyl acids as products, depending on the identity of R in the formula above.

Accordingly, the invention further refers to a method for cleaving the substrate molecule of formula I, comprising the steps of:

providing the substrate molecule;

contacting the substrate molecule with an amount of a polypeptide described herein to cleave the substrate molecule.

“Suitable conditions” for contacting the substrate molecule and the polypeptide include any conditions appropriate for the reaction to occur, such as the presence of citryl-CoA, 10 mM dithiothreitol, 10 mM MgCl₂, in a suitable buffer, e.g. 100 mM Tris-HCl (pH 8.5).

Still further, the invention refers to a kit for carrying out the method above, which kit comprises, in separate vials, the substrate molecule and the Cit E polypeptide, and optionally any other suitable reagents. By suitable reagents are meant such molecules or chemicals, which allows a suitable condition. This can include Citryl-CoA sodium salt or malyl-CoA sodium salt, a suitable reaction buffer, enzymatic reagents to enable a spectrophotometric readout such as malate dehydrogenase and lactate dehydrogenase together with NADH. The oxidation by NADH can be measured in a spectrophotometer at 340 nm wavelength. Alternatively, the formation of acetyl-CoA in the enzymatic reaction can be measured by reverse-phase high-performance liquid chromatography.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Below, the invention is described in the appended examples, which are intended to illustrate the invention, without limiting the scope of protection.

### EXAMPLES

### Example 1

### Identification of a Mouse EST Homologous to C. elegans Hypothetical Protein C01G10.7 and Bacterial Citryl-CoA Lyase (CitE Gene Product)

Male C57B1/6J mice, 12 weeks old, were housed under conditions of ambient temperature and free access to water and chow. Either the β3-selective agonist CL 316,243 (1mg/kg) or saline was injected every 12h with 2 doses during a total period of 24 hours. The animals were decapitated 24 h after the experiment was started, and the white adipose tissue was quickly collected and immediately frozen into liquid nitrogen.

PolyA⁺ mRNAs were extracted from white adipose tissue from control and β3-agonist treated mice respectively. They were reverse transcribed using a T7-tagged oligo-dT primer and double-stranded cDNAs were generated. These cDNAs were then amplified and labeled using In vitro transcription (IVT) with T7 RNA polymerase and biotinylated nucleotides. The populations of cRNAs obtained after IVT were purified and fragmented by heat to produce a distribution of RNA fragment sizes from approximately 35 to 200 bases. Two Affymetrix (Affymetrix, Inc., 3380 Central Exwy, Santa Clara, Calif. 95051) Mu19K and Mu11K sets of 3 arrays (subA, subB and subC) and 2 arrays (subA and subB) respectively, were hybridized using the recommended buffer overnight at 45° C. with the control or the treated denatured samples. The arrays were then washed and stained with R-phycoerythrin streptavidin with the help of an Affymetrix fluidics station. The cartridges were scanned using a Hewlett-Packard confocal scanner and the images were analyzed with the GeneChip 3.1 software (Affymetrix). The identity of the genes represented on the Affymetrix GeneChips was assessed by performing searches using BLAST (Altschul et al., J. Molec. Biol., 1990, 215, 403-410), which is incorporated herein by reference in its entirety) on available protein sequence databanks. One of the mouse EST clusters represented on the Mu19K set of GeneChips that was found to correspond to a gene transcriptionally downregulated by a factor of two (TIGR Mouse Gene Index TC20248) showed a significant homology with a C. elegans hypothetical protein C01G10.7 (GenBank™ Acc.# CAB02709). The sequence of protein C01G10.7 was in turn used as query sequence BLAST searches against available protein sequence databanks and found to have significant homology with Leuconostoc mesenteroides citrate lyase β-chain (citryl-CoA lyase; encoded by CitE).

The nucleotide sequence of TC20248 was used as query sequence for BLAST searches of the Incyte Genomics (Incyte Genomics, Inc., 3160 Porter Drive, Palo Alto, Calif. 94304) mouse Expressed Sequence Tag database (ZooSeq). Several highly scoring sequences were detected.

### Example 2

### Cloning of Rat CitE Homolog cDNA

The nucleotide and translated amino acid sequences of the mouse CitE homolog was used as query sequences in a BLAST run using the Incyte ZooSeq rat EST database. The sequence from the following Incyte clones #702234264/701883631/701034392 were used to design oligonucleotide primers for cDNA cloning by RACE. The full length sequence was obtained by performing both 5′ and 3′ RACE (c.f. Zhang Y, Frohman M A, Methods Mol Biol 1997;69:61-87) on rat brain cDNA. The respective sequences of the primers were:

[SEQ ID NO:7]

5′RACE primer (332): 5′-AAGCTGCCCTGGGTACTGTGGTTTG

-3′
[SEQ ID NO:8]

3′RACE primer (207): 5′-CAGATTGCCGTGGTACAGGAACAGT

-3′

For isolation of the entire cDNA encoding rat CitE homolog, the mRNA substrate was prepared by using the mRNA Direct kit from Dynal AS to prepare rat polyA+ RNA from hypothalamus. The manufacturer's protocol was followed completely. Three hypothalami from normal Sprague-Dawley rats were used for mRNA preparation.

AltI:

The RACE reactions were performed using GeneRacer from Invitrogen (cat #L1500-01). The manufacturers manual was followed completely except for precipitations that were performed using pellet paint co-precipitant (Sigma, 69049-3) The RACE PCR amplifications were performed as follows; 5 cycles of 94° C., 30 sec; 72° C., 3 min was followed by 5 cycles of 94° C., 30 sec; 70° C., 30 sec; 72° C., 3 min, and 22 cycles of 94° C., 30 sec; 68° C., 30 sec; 72° C., 3 min, and resulted in fragments of approximately 1200 and 500 bp for the 5′- and 3′-RACE respectively.

AltII:

The RACE reactions were performed using SMART RACE cDNA Amplification Kit from Clontech (#K1811-1). The manufacturers manual was followed completely with the following modifications: (1) PolyA+ RNA was used instead of total RNA; (2) The first RACE-PCR utilized the 3′RACE primer and the 5′RACE primer for 3′ and 5′RACE, respectively. The RACE PCR amplifications were performed as shown below and resulted in a band of approximately 500 bp for the 3′-RACE reaction. 5 cycles of 94° C., 30 sec; 72° C., 3 min was followed by 5 cycles of 94° C., 30 sec; 70° C., 30 sec; 72° C., 3 min, and 22 cycles of 94° C., 30 sec; 68° C., 30 sec; 72° C., 3 min.

The fragments were gel purified using QIAquick Gel Extraction Kit (#1.6122-50 from Qiagen). The manufacturers protocol was used and samples were eluted in 30 μl elution buffer. The products were cloned into pCRII-TOPO vector using TOPO TA Cloning Kit (#K4600-01, Invitrogen). Transformants were picked with a toothpick to 100 μl water. The toothpicks were then transferred to Terrific Broth (#22711-022, Life Technologies) containing 60 μg/ml ampicillin (#Q100-16, Invitrogen) for making glycerol stocks. The bacterial colonies were transferred to water and boiled for 5 min to release plasmid DNA and 10 μl extract was used in colony PCR with vector specific oligonucleotide primers as follows:

[SEQ ID NO:9]

Forward primer (na 11): 5′-CACAGGAAACAGCTATGAC-3′

[SEQ ID NO:10]

Reverse primer (na 10): 5′-CCAGTCACGACGTTGTAAA-3′

10 μl boiled colony extract was mixed together with 2 μl forward vector primer, 2 μl reverse vector primer, 5 μl 10×PCR buffer (provided with the enzyme, Amersham Pharmacia Biotech), 0.5 μl 10 mM dNTP (#1 969 064 from Boehringer Mannheim), 0.5 μl Taq polymerase (#27-0799-62, Amersham Pharmacia Biotech) and 30 μl water.

Cycle conditions in MJ research Tetrad Thermocycler were as follows: Heating the sample to 94° C. for 5 minutes, followed by 30 cycles of 94° C., 1 min; 50° C., 1 min; 72° C., 3 min, followed by maintaining the sample at 72° C. for 10 min.

Preparation of plasmids from a total of 9 clones was done using QIAprep Spin Miniprep Kit (#1.6103-50) from Qiagen. Seven clones were sequenced using Dye Terminator Cycle Sequencing with AmpliTaq—DNA Polymerase FS and ABI377XL Sequencer and all corresponded to rat CitE homolog. The entire sequence of the rat CitE homolog could be assembled after sequencing of above clones (SEQ ID NO: 3) into a continuous nucleotide sequence of 1240 base-pairs containing an open reading frame of 338 amino acids (SEQ ID NO: 4).

### Example 3

### Cloning of Mouse CitE Homolog cDNA

The nucleotide sequence of TC20248 representing EST sequences emanating from a mouse gene encoding a homolog of the C. elegans hypothetical protein C01G10.7 was used as a query sequence in a BLAST run against the GenBank™ mouse EST database in order to retrieve additional EST sequences emanating from this gene and to design oligonucleotides for RACE (Rapid Amplification of cDNA Ends; Matz, M., et al. (1999) Nucleic Acids Res. 27: 1558-1560). The nucleotide sequence of IMAGE clones #1450860/1889749/1890093 was used to design RACE primers for cloning of the entire part of the cDNA:

[SEQ ID NO:11]

3′RACE primer (209) 5′-TTGTGCGTGCTGCGGAACACGGT-3′

[SEQ ID NO:12]

5′RACE primer (210) 5′-GCAGTCCACACGGCACAATCTACT-3′

The RACE was performed using SMART RACE cDNA Amplification Kit from Clontech (#K1811-1). Mouse brain or mouse kidney polyA+ RNA (#6616-1 and #6613-1, Clontech) was used as starting material. The manufacturer's manual was followed completely with the following modifications: (1) PCR cycling in 0.2 ml tubes in MJ research Tetrad Thermocycler using the following program: 5 cycles of 94° C., 30 sec; 72° C., 3 min was followed by 5 cycles of 94° C., 30 sec; 70° C., 30 sec; 72° C., 3 min, and 25 cycles of 94° C., 30 sec; 68° C., 30 sec; 72° C., 3 min.

The RACE on brain cDNA resulted in products of approximately 1600 and 600 base pairs in length for the 3′- and 5′-RACE, respectively. Using kidney cDNA in the reaction resulted in a 600 bp fragment for the 5′-RACE reaction. Gel purification of amplified products using QIAquick Gel Extraction Kit (#1.6122-50, Qiagen) was performed following the provided protocol. After purification, the fragments were immediately ligated to pCRII-TOPO vector and transformed into TOP10-cells, using the TOPO TA cloning kit from Promega (#K4600-01). Colony PCR of the resulting bacterial transformants was performed as described above according to the following scheme: heating the sample to 94° C. for 5 min; 35 cycles of 94° C., 1 min; 50° C., 1 min; 72° C., 2 min, followed by maintaining the sample at 72° C. for 10 min.

The positive clones were subject to preparation of plasmid using QIAprep Spin Miniprep Kit (#1.6103-50) from Qiagen and sequenced as described above. The complete nucleotide sequence of the fragments could be assembled with the nucleotide sequence of IMAGE clones #1450860/1889749/1890093 with perfect sequence match (SEQ ID NO: 5). The consensus resulted in a continuous nucleotide sequence of 1286 base-pairs (SEQ ID NO: 5) containing an open reading frame of 338 amino acids (SEQ ID NO: 6) defining the mouse CitE homolog.

### Example 4

### Identification of Human CitE Homolog

The Incyte Genomics (Incyte Genomics, Inc., 3160 Porter Drive, Palo Alto, Calif. 94304) human Expressed Sequence Tag database (LifeSeq Gold) was searched using BLASTP (Altschul et al., J. Molec. Biol., 1990, 215, 403-410, which is incorporated herein by reference in its entirety), using the mouse CitE homolog as a query sequence. One highly scoring sequence was detected (Sequence ID 7596666H1) that putatively contained the entire open reading frame of the human CitE homolog. Physical plasmid DNA corresponding to this sequence was obtained from Incyte Genomics and entire nucleotide sequence was determined of the plasmid insert using standard methods well known in the art, and the resulting sequence is given as SEQ ID NO: 1.

### Example 5

### Northern Blot Analysis of Rat CitE Homolog mRNA

Northern blot hybridizations were performed to examine the expression of rat CitE homolog mRNA. The sequence information obtained as described in Example 2 was used to design PCR primers (see below) to be used for probe generation. The PCR was performed as follows:

[SEQ ID NO:8]

Forward PCR primer (207) 5′-CAGATTGCCGTGGTACAGGAAC

AGT-3′
[SEQ ID NO:7]

Reverse PCR primer (332) 5′-AAGCTGCCCTGGGTACTGTGGT

TTG-3′

0.25 μl plasmid of 3′RACE product was mixed with 1.5 μl fwd primer, 1.5 μl rev primer, 5 ∥l 10×buffer (provided with the enzyme), 1 μl 10 mM dNTP (#1 969 064 from Boehringer Mannheim), 0.5 μl Herculase polymerase (#600262, AH Diagnostics), and 40.25 μl water.

PCR was performed in a MJ Research PTC-100 Thermocycler using the following program; heating the sample at 94° C. for 5 min, followed by 26 cycles of 94° C., 1 min; 55° C., 1 min; 72° C., 1 min, followed by maintaining the sample at 72° C. for 10 min.

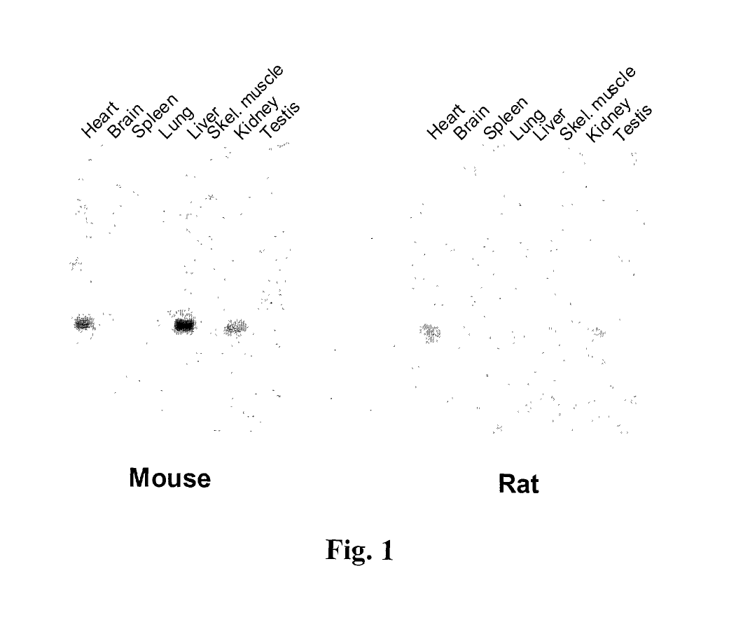

The PCR product of 300 bp was purified using QIAquick PCR Purification Kit (#28104) from Qiagen, and radioactively labeled with 32P-dATP (#AA0004/250, Amersham Pharmacia Biotech) was done by random priming using “Strip-EZ DNA Stripable DNA probe synthesis and removal kit” (#1470) from Ambion. Hybridization was carried out on Rat Multiple Tissue Northern Blot (MTN, #7764-1) from Clontech as described in manufacturer's protocol. After exposure overnight on Molecular Dynamics Phosphor Imager screen (#MD146-814) bands of about 1.5 kb were visible in the heart, kidney, and spleen lanes as well as weakly in the lanes for brain, skeletal muscle and testis (FIG. 1).

### Example 6

### Northern Blot Analysis of Mouse CitE Homolog mRNA

Northern blot hybridizations were performed to examine the expression of mouse CitE homolog mRNA. The sequence information obtained as described in Example 3 was used to design PCR primers (see below) to be used for probe generation. The PCR was performed as follows:

[SEQ ID NO:11]

Forward PCR primer (209) 5′-TTGTGCGTGCTGCGGAACACGG

T-3′
[SEQ ID NO:12]

Reverse PCR primer (210) 5′-GCAGTCCACACGGCACAATCTA

CT-3′

0.25 μl plasmid of 5′RACE product was mixed with 1.5 μl fwd primer, 1.5 μl rev primer, 5 μl 10×buffer (provided with the enzyme), 1 μl 10 mM dNTP (#1 969 064 from Boehringer Mannheim), 0.5 μl Herculase polymerase (#600262, AH Diagnostics), and 40.25 μl water.

PCR was performed in a MJ Research PTC-100 Thermocycler using the following program; heating the sample at 94° C. for 5 min, followed by 26 cycles of 94° C., 1 min; 55° C., 1 min; 72° C., 1 min, followed by maintaining the sample at 72° C. for 10 min.

The PCR product of 200 bp was purified using QIAquick PCR Purification Kit (#28104) from Qiagen, and radioactively labeled 32P-dATP (#AA0004/250, Amersham Pharmacia Biotech) was done by random priming using “Strip-EZ DNA Stripable DNA probe synthesis and removal kit” (#1470) from Ambion. Hybridization was carried out on Rat Multiple Tissue Northern Blot (MTN, #7764-1) from Clontech as described in manufacturer's protocol. After exposure overnight on Molecular Dynamics Phosphor Imager screen (#MD146-814) bands of about 1.4 kb were visible in the heart, kidney, and liver lanes as well as weakly in the lanes for brain, lung, skeletal muscle and testis (FIG. 1).

### Example 7

mRNA blot hybridizations were performed to examine the expression of human CitE homolog mRNA. The sequence information obtained as described in example 4 was used to design PCR primers (see below) to be used for probe generation. The PCR was performed as follows:

(SEQ ID NO:13)

Forward PCR primer 5′-TGGGCCCTACTGAAAAATGTGTGAG-3′

(SEQ ID NO:14)

Reverse PCR primer 5′-GGCGGCTCCTTCTCGTGACT-3′

0.25 μl of Incyte clone # 7596666 was mixed with 1.5 μl fwd primer, 1.5 μl rev primer, 5 μl 10×buffer (provided with the enzyme), 1 μl 10 mM dNTP (#1 969 064 from Boehringer Mannheim), 0.5 μl Taq polymerase (#27-0799-62, Amersham Pharmacia Biotech), and 40.25 μl water.

PCR was performed in a MJ Research PTC-100 Thermocycler using the following program: heating the sample at 94° C. for 5 min, followed by 25 cycles of 94° C., 1 min; 60° C., 1 min; 72° C., 1 min, followed by maintaining the sample at 72° C. for 10 min.

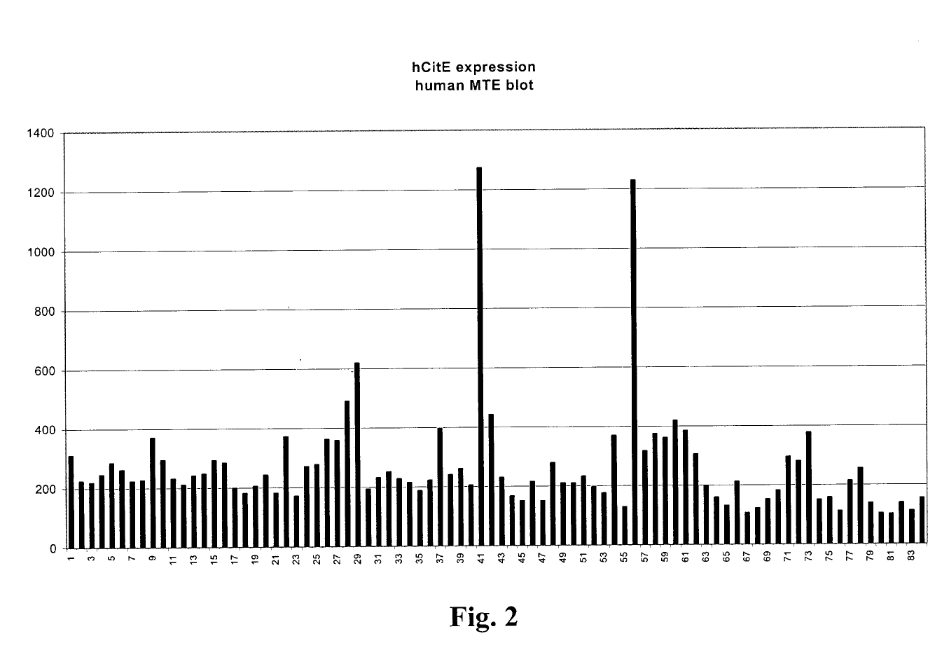

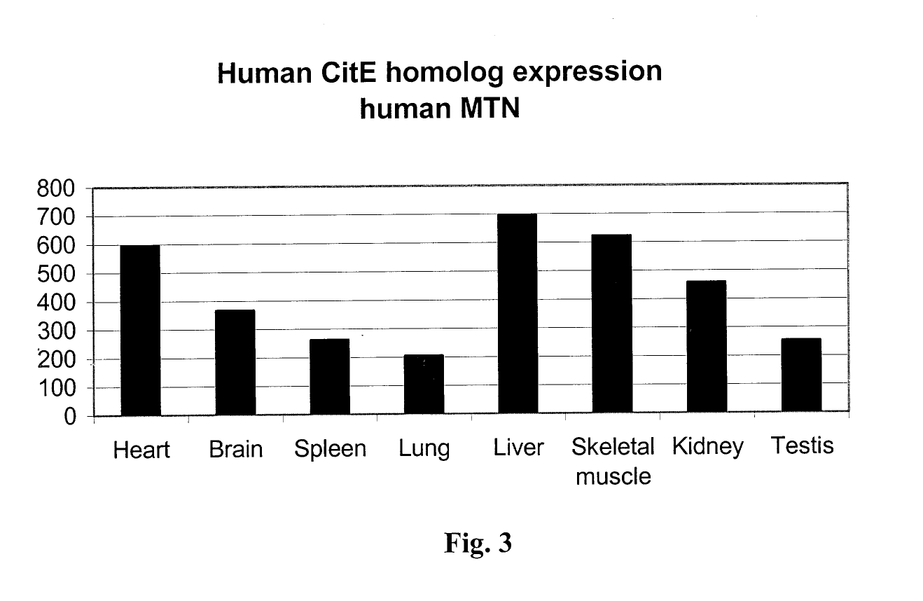

The PCR product of 502 bp was purified using QIAquick PCR Purification Kit (#28104) from Qiagen, and radioactively labeled with 32P-dATP (#AA0004/250, Amersham Pharmacia Biotech) was done by random priming using “Strip-EZ DNA Stripable DNA probe synthesis and removal kit” (#1470) from Ambion. Hybridization was carried out on human Multiple Tissue Expression Array (MTE, #7775-1) and human Multiple Tissue Northern (#7760-1) from Clontech as described in manufacturer's protocols. After exposure overnight on Molecular Dynamics Phosphor Imager screen (#MD146-814) dots were visible in the heart, kidney, and spleen squares as well as weakly in skeletal muscle on the MTE filter (FIG. 2; Table 1). The MTN resulted in hybridization in lanes of heart, liver, kidney and skeletal muscle and maybe weakly in brain and testis (FIG. 3).

TABLE 1
Tissues analyzed in Multiple Tissue Expression

| number | Name |
|---|---|
| 1 | Whole brain |
| 2 | cerebral cortex |
| 3 | frontal lobe |
| 4 | parietal lobe |
| 5 | occipital lobe |
| 6 | temporal lobe |
| 7 | pg of cerebral cortex |
| 8 | pons |
| 9 | cerebellum, left |
| 10 | cerebellum, right |
| 11 | corpus callosum |
| 12 | amygdala |
| 13 | caudate nucleus |
| 14 | hippocampus |
| 15 | medulla oblongata |
| 16 | putamen |
| 17 | subst nigra |
| 18 | accummbens |
| 19 | thalamus |
| 20 | pituitary |
| 21 | spinal cord |
| 22 | heart |
| 23 | aorta |
| 24 | atrium, left |
| 25 | atrium, right |
| 26 | ventricle, left |
| 27 | ventricle, right |
| 28 | interventricular septum |
| 29 | apex of the heart |
| 30 | esophagus |
| 31 | stomach |
| 32 | duodenum |
| 33 | jejunum |
| 34 | ileum |
| 35 | iloceum |
| 36 | appendix |
| 37 | colon, ascending |
| 38 | colon, transverse |
| 39 | colon, descending |
| 40 | rectum |
| 41 | kidney |
| 42 | skeletal muscle |
| 43 | spleen |
| 44 | thymus |
| 45 | peripheral blood |
| | leukocyte |
| 46 | lymph node |
| 47 | bone marrow |
| 48 | trachea |
| 49 | lung |
| 50 | placenta |
| 51 | bladder |
| 52 | uterus |
| 53 | prostate |
| 54 | testis |
| 55 | ovary |
| 56 | liver |
| 57 | pancreas |
| 58 | adrenal gland |
| 59 | thyroid gland |
| 60 | salivary gland |
| 61 | mammary gland |
| 62 | leukemis, HL-60 |
| 63 | HeLa S3 |
| 64 | leukemia, K-562 |
| 65 | leukemia, MOLT-4 |
| 66 | Raji |
| 67 | Daudi |
| 68 | SW480 |
| 69 | A549 |
| 70 | fetal brain |
| 71 | fetal heart |
| 72 | fetal kidney |
| 73 | fetal liver |
| 74 | fetal spleen |
| 75 | fetal thymus |
| 76 | fetal lung |
| 77 | yeast to RNA |
| 78 | yeast tRNA |
| 79 | E coli rRNA |
| 80 | Ecoli DNA |
| 81 | Poly r(A) |
| 82 | human COT-1 DNA |
| 83 | human DNA 100 ng |
| 84 | human DNA 500 ng |

### Example 8

### Recombinant Expression of hCitE in Eukaryotic Host Cells

A. Expression of hCitE in Mammalian Cells

To produce hCitE protein, a hCitE-encoding polynucleotide is expressed in a suitable host cell using a suitable expression vector and standard genetic engineering techniques. For example, the hCitE-encoding sequence described in Example 4 is subcloned into the commercial expression vector pzeoSV2 (Invitrogen, San Diego, Calif.) and transfected into Chinese Hamster Ovary (CHO) cells using the transfection reagent FuGENE6™ (Boehringer-Mannheim) and the transfection protocol provided in the product insert. Other eukaryotic cell lines, including human embryonic kidney (HEK 293) and COS cells, are suitable as well. Cells stably expressing hCitE are selected by growth in the presence of 100 μg/ml zeocin (Stratagene, LaJolla, Calif.). Optionally, hCitE may be purified from the cells using standard chromatographic techniques. To facilitate purification, antisera is raised against one or more synthetic peptide sequences that correspond to portions of the hCitE amino acid sequence, and the antisera is used to affinity purify hCitE protein. The hCitE also may be expressed in-frame with a tag sequence (e.g., polyhistidine, hemagluttinin, FLAG) to facilitate purification. Moreover, it will be appreciated that many of the uses for hCitE polypeptides, such as assays described below, do not require purification of hCitE from the host cell.

B. Expression of hCitE in Insect Cells

For expression of hCitE in a baculovirus system, a polynucleotide molecule having a nucleotide sequence of SEQ ID NO: 1 can be amplified by PCR. The forward primer is determined by routine procedures and preferably contains a 5′ extension which adds the NdeI cloning site, followed by nucleotides which correspond to a nucleotide sequence of SEQ ID NO: 1. The reverse primer is also determined by routine procedures and preferably contains a 5′ extension which introduces the KpnI cloning site, followed by nucleotides which correspond to the reverse complement of a nucleotide sequence of SEQ ID NO: 1.

The PCR product is gel purified, digested with NdeI and KpnI, and cloned into the corresponding sites of vector pACHTL-A (Pharmingen, San Diego, Calif.). The pAcHTL expression vector contains the strong polyhedrin promoter of the Autographa californica nuclear polyhedrosis virus (AcMNPV), and a 6×His tag upstream from the multiple cloning site. A protein kinase site for phosphorylation and a thrombin site for excision of the recombinant protein precede the multiple cloning site is also present. Of course, many other baculovirus vectors could be used in place of pAcHTL-A, such as pAc373, pVL941 and pAcIM1. Other suitable vectors for the expression of hCitE polypeptides can be used, provided that the vector construct includes appropriately located signals for transcription, translation, and trafficking, such as an in-frame AUG and a signal peptide, as required. Such vectors are described in Luckow et al., Virology 170:31-39, among others.

The virus is grown and isolated using standard baculovirus expression methods, such as those described in Summers et al. (A Manual of Methods for Baculovirus Vectors and Insect Cell Culture Procedures, Texas Agricultural Experimental Station Bulletin No. 1555 (1987)).

In a preferred embodiment, pAcHLT-A containing hCitEgene is introduced into baculovirus using the “BaculoGold™” transfection kit (Pharmingen, San Diego, Calif.) using methods established by the manufacturer. Individual virus isolates are analyzed for protein production by radiolabeling infected cells with ³⁵S-methionine at 24 hours post infection. Infected cells are harvested at 48 hours post infection, and the labeled proteins are visualized by SDS-PAGE. Viruses exhibiting high expression levels can be isolated and used for scaled up expression.

For expression of a hCitE polypeptide in a Sf9 cells, a polynucleotide molecule having the nucleotide sequence of SEQ ID NO: 1 can be amplified by PCR using the primers and methods described above for baculovirus expression. The hCitE cDNA is cloned into vector pAcHLT-A (Pharmingen) for expression in Sf9 insect. The insert is cloned into the NdeI and KpnI sites, after elimination of an internal NdeI site (using the same primers described above for expression in baculovirus). DNA is purified with Qiagen chromatography columns and expressed in Sf9 cells. Preliminary Western blot experiments from non-purified plaques are tested for the presence of the recombinant protein of the expected size, which reacted with the hCitE-specific antibody. These results are confirmed after further purification and expression optimization in HiG5 cells.

### Example 9

### Antibodies to hCitE

Standard techniques are employed to generate polyclonal or monoclonal antibodies to the hCitE protein, and to generate useful antigen-binding fragments thereof or variants thereof, including “humanized” variants. Such protocols can be found, for example, in Sambrook et al. (1989) and Harlow et al. (Eds.), Antibodies A Laboratory Manual; Cold Spring Harbor Laboratory; Cold Spring Harbor, N.Y. (1988). In one embodiment, recombinant hCitE polypeptides (or cells or cell membranes containing such polypeptides) are used as antigen to generate the antibodies. In another embodiment, one or more peptides having amino acid sequences corresponding to an immunogenic portion of hCitE protein (e.g., 6, 7, 8, 9, 10, 11, 12, 13, 14, 15, 16, 17, 18, 19, 20, or more amino acids) are used as antigen. The antigen may be mixed with an adjuvant or linked to a hapten to increase antibody production.

As one exemplary protocol, recombinant hCitE or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of hCitE antigen are emulsified with Freund's Incomplete Adjuvant and injected subcutaneously. Prior to the final booster injection, a serum sample is taken from the immunized mice and assayed by western blot to confirm the presence of antibodies that immunoreact with hCitE. Serum from the immunized animals may be used as polyclonal antisera or used to isolate polyclonal antibodies that recognize hCitE. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged, resuspended in RPMI containing 15% FBS, 100 μM sodium hypoxanthine, 0.4 μM aminopterin, 16 μM thymidine (HAT) (Gibco), 25 units/ml IL-6 (Boehringer-Mannheim) and 1.5×10⁶ thymocytes/ml, and plated into 10 Corning flat-bottom 96-well tissue culture plates (Corning, Corning N.Y.).

On days 2, 4, and 6 after the fusion, 100 μl of medium is removed from the wells of the fusion plates and replaced with fresh medium. On day 8, the fusions are screened by ELISA, testing for the presence of mouse IgG that binds to hCitE. Selected fusion wells are further cloned by dilution until monoclonal cultures producing anti-hCitE antibodies are obtained.

### Example 10

### Interaction Trap/Two-Hybrid System

In order to assay for hCitE-interacting proteins, the interaction trap/two-hybrid library screening method can be used. This assay was first described in Fields et al., Nature, 1989, 340, 245, which is incorporated herein by reference in its entirety. A protocol is published in Current Protocols in Molecular Biology 1999, John Wiley & Sons, NY and Ausubel, F. M. et al. 1992, Short protocols in molecular biology, fourth edition, Greene and Wiley-interscience, NY, which is incorporated herein by reference in its entirety. Kits are available from Clontech, Palo Alto, Calif. (Matchmaker Two-Hybrid System 3).

A fusion of the nucleotide sequences encoding all or partial hCitE and the yeast transcription factor GAL4 DNA-binding domain (DNA-BD) is constructed in an appropriate plasmid (i.e. pGBKT7) using standard subcloning techniques. Similarly, a GAL4 active domain (AD) fusion library is constructed in a second plasmid (i.e. pGADT7) from cDNA of potential hCitE-binding proteins (for protocols on forming cDNA libraries, see Sambrook et al. 1989, Molecular cloning: a laboratory manual, second edition, Cold Spring Harbor Press, Cold Spring Harbor, N.Y., which is incorporated herein by reference in its entirety). The DNA-BD/hCitE fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 105 transformants/mg DNA) with both the hCitE and library fusion plasmids according to standard procedure (Ausubel, et al., 1992, Short protocols in molecular biology, fourth edition, Greene and Wiley-interscience, NY, which is incorporated herein by reference in its entirety). In vivo binding of DNA-BD/hCitE with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-3-indolyl-β-D-galactoside) supplemented media (filter assay for β-galactosidase activity is described in Breeden et al., Cold Spring Harb. Symp. Quant. Biol., 1985, 50, 643, which is incorporated herein by reference in its entirety). Positive AD-library plasmids are rescued from transformants and reintroduced into the original yeast strain as well as other strains containing unrelated DNA-BD fusion proteins to confirm specific hCitE/library protein interactions. Insert DNA is sequenced to verify the presence of an open reading frame fused to GAL4 AD and to determine the identity of the hCitE-binding protein.

### Example 11

### Assay for Modulators of hCitE Enzyme Activity

Set forth below is an example nonlimiting assay for identifying modulators (inhibitors and enhancers) of hCitE activity. Among the modulators that can be identified by this assay are natural substrate, enhancing or inhibitory compounds of hCitE; synthetic analogs and derivatives of natural substrates, enhancers or inhibitors; antibodies, antibody fragments, and/or antibody-like compounds derived from natural antibodies or from antibody-like combinatorial libraries; and/or synthetic compounds identified by high-throughput screening of libraries; and the like. All modulators that bind or modulate hCitE are useful for identifying hCitE activity in tissue samples (e.g., for diagnostic purposes, pathological purposes, and the like). Enhancing and inhibitory modulators are useful for up-regulating and down-regulating hCitE activity, respectively, to treat disease states characterized by abnormal levels of hCitE activity. The assays may be performed using single putative modulators, and/or may be performed using a known enhancer in combination with candidate inhibitor (or visa versa).

hCitE activity can be measured by the malate dehydrogenase-catalyzed reduction of oxaloacetate by NADH (modified from: Gribble, A. D., et al. (1996) J. Med. Chem. 39, 3569-3584). The standard reaction mixture (100 μl) contains 100 mM Tris-HCl, pH 8.5, 10 mM MgCl₂, 10 mM dithiothreitol, 0.14 mM NADH, 2 units of malate dehydrogenase, 5 mM citryl-CoA. For kinetic analysis of hCitE, a sample of the freshly thawed recombinant protein in a purified form or as a cell-extract (see above) is preincubated with enhancing or inhibitory compounds for 90 min at 4° C. The reaction is started by the addition of 1 μl of the enzyme preparation to the reaction mixture, and the rate of NADH oxidation can be measured at 340 nm with a spectrophotometer. One unit of enzyme can be defined as the amount of enzyme that oxidizes 1 μmol of NADH per min at 25° C.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. An isolated nucleic acid molecule comprising a nucleotide sequence that is at least 85% identical to the sequence of SEQ ID NO: 1, SEQ ID NO: 3, or SEQ ID NO: 5.

2. The nucleic acid molecule of claim 1, wherein the nucleotide sequence encodes a polypeptide having citrate lyase activity.

3. The nucleic acid molecule of claim 1, wherein the nucleic acid molecule comprises the sequence of SEQ ID NO: 1, SEQ ID NO: 3, or SEQ ID NO: 5.

4. An isolated nucleic acid molecule comprising a nucleotide sequence that encodes a polypeptide comprising an amino acid sequence that is at least 85% identical to the sequence of SEQ ID NO: 2, SEQ ID NO: 4, or SEQ ID NO: 6.

5. The nucleic acid molecule of claim 4, wherein the polypeptide has citrate lyase activity.

6. An isolated nucleic acid comprising a nucleotide sequence that encodes a polypeptide comprising the amino acid sequence of SEQ ID NO: 2, SEQ ID NO: 4, or SEQ ID NO: 6.

7. An isolated nucleic acid molecule comprising a nucleotide sequence that hybridizes under stringent hybridization conditions to the sequence of SEQ ID NO: 1, SEQ ID NO: 3, SEQ ID NO: 5, or a complete complement thereof.

8. The nucleic acid molecule of claim 7, wherein the nucleotide sequence encodes a polypeptide having citrate lyase activity.

9. An isolated nucleic acid molecule comprising a nucleotide sequence that encodes a polypeptide comprising at least 100 contiguous amino acid residues of SEQ ID NO: 2, SEQ ID NO: 4, or SEQ ID NO: 6.

10. An isolated nucleic acid molecule comprising a nucleotide sequence that encodes a polypeptide comprising an immunogenic fragment of at least 20 amino acids of SEQ ID NO: 2, SEQ ID NO: 4, or SEQ ID NO: 6.

11. A substantially pure polypeptide comprising an amino acid sequence that is at least 85% identical to the sequence of SEQ ID NO: 2, SEQ ID NO: 4, or SEQ ID NO: 6.

12. The polypeptide of claim 11, wherein the polypeptide has citrate lyase activity.

13. A substantially pure polypeptide comprising the amino acid sequence of SEQ ID NO: 2, SEQ ID NO: 4, or SEQ ID NO: 6.

14. The polypeptide of claim 13, wherein the polypeptide consists of the amino acid sequence of SEQ ID NO: 2, SEQ ID NO: 4, or SEQ ID NO: 6.

15. A substantially pure polypeptide comprising at least 100 contiguous amino acid residues of SEQ ID NO: 2, SEQ ID NO: 4, or SEQ ID NO: 6.

16. A substantially pure polypeptide comprising an immunogenic fragment of at least 20 amino acids of SEQ ID NO: 2, SEQ ID NO: 4, or SEQ ID NO: 6.

17. A vector comprising the nucleic acid molecule of claim 1.

18. A replicable expression vector comprising the nucleic acid molecule of claim 1 operably linked to a regulatory element that directs expression of the nucleic acid molecule.

19. A cultured host cell comprising the vector of claim 17.

20. A method for producing a polypeptide, the method comprising culturing the host cell of claim 19 under conditions whereby the polypeptide is produced.

21. A nucleic acid probe comprising at least 15 nucleotides, wherein the probe specifically hybridizes to at least a part of the nucleic acid molecule of claim 3, said part having a sequence shown as in SEQ ID NO: 1, SEQ ID NO: 3, or SEQ ID NO: 5.

22. An antisense oligonucleotide having a sequence that specifically hybridizes to at least a part of the nucleic acid molecule of claim 3, said part having a sequence shown as in SEQ ID NO: 1, SEQ ID NO: 3, or SEQ ID NO: 5.

23. A pair of primers comprising a first primer and a second primer, wherein the first primer hybridizes to the sense strand of the nucleic acid molecule of claim 3, and wherein the second primer hybridizes to a strand complementary to the sense strand of the nucleic acid molecule.

24. An isolated antibody that specifically binds to the polypeptide of claim 14.

25. A method for screening for a compound that modulates an activity of a citrate lyase, the method comprising:
contacting the polypeptide of claim 11 with a test compound;
measuring a citrate lyase activity of the polypeptide in the presence of the test compound;
comparing the citrate lyase activity of the polypeptide in the presence of the test compound with the citrate lyase activity of the polypeptide in the absence of the test compound,
to thereby determine whether the test compound modulates an activity of a citrate lyase.

26. A kit for carrying out the method of claim 25, the kit comprising a substantially pure polypeptide comprising an amino acid sequence that is at least 85% identical to the sequence of SEQ ID NO: 2, SEQ ID NO: 4, or SEQ ID NO: 6, and instructions for use.

27. A method of identifying an agent that binds to a citrate lyase, the method comprising:
contacting a polypeptide comprising the amino acid sequence of SEQ ID NO: 2, SEQ ID NO: 4, or SEQ ID NO: 6 with a candidate agent; and
determining that the candidate agent binds to the polypeptide,
to thereby identify a candidate agent that binds to a citrate lyase.

28. A method for disrupting a citrate lyase (Cit E) gene in a non-human embryonic stem cell, the method comprising:
providing a nucleotide sequence capable of disrupting a Cit E gene; and
introducing the nucleotide sequence into a non-human embryonic stem-cell under conditions such that nucleotide sequence is homologously recombined into the Cit E gene in the genome of the cell, to produce a cell containing at least one disrupted Cit E allele.

29. A non-human transgenic animal expressing reduced levels of Cit E, wherein the Cit E gene has been disrupted by the method of claim 28.

30. A non-human transgenic animal whose genome comprises an antisense nucleic acid molecule that hybridizes to an mRNA encoding the polypeptide of claim 4, thereby reducing translation of the polypeptide in the animal.

31. The non-human transgenic animal of claim 30, wherein the animal is a mouse.

32. The non-human transgenic animal of claim 30, wherein the animal is of the species Caenorhabditis elegans.

33. A method for screening for compounds that affect a fatty acid metabolism pathway, the method comprising:
providing the non-human transgenic animal of claim 29;
providing a composition comprising a test compound in a form suitable for administration to the non-human animal;
administering the test compound to the non-human animal; and
determining the effect of the test compound on a fatty acid metabolism pathway in the animal.

34. A method for screening for compounds that modulate citrate lyase activity, the method comprising:
contacting a test compound to a citrate lyase;
determining the effect of the test compound on a citrate lyase activity of the citrate lyase;
contacting the test compound to the polypeptide of claim 11; and
determining the effect of the test compound on a citrate lyase activity of the polypeptide,
to thereby screen for compounds that modulate citrate lyase activity.

35. A method for the treatment of a disorder selected from the group consisting of type 2 diabetes, type 1 diabetes, maturity-onset diabetes of the young (MODY), gestational diabetes, obesity, dyslipidemia, hypertension, cancer, cardiovascular disease, digestive disease, respiratory disease, neuropathy, nephropathy, retinopathy, hypertension, and atherosclerosis, the method comprising administering to an individual in need thereof an amount of an agent effective to modulate the expression or activity of a Cit E polypeptide.

36. A substrate molecule for CitE, which substrate molecule is represented by formula I: 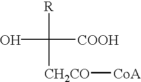
wherein R is CH₂COOH, H, CH₃, CH₃(CH₂)ₙ or (CH₂)ₘCOOH, whereby n is 1, 2, 3, 4, 5 or 6, and m is 2, 3, 4, 5, 6 or 7.

37. A method for cleaving a substrate molecule, the method comprising:
providing the substrate molecule of claim 36; and
contacting the substrate molecule with an amount of the polypeptide of claim 11 to cleave the substrate molecule.

38. A kit for carrying out the method of claim 37, the kit comprising, in separate vials, the substrate molecule and the polypeptide.


SEQUENCE LISTING (14 sequences)

SEQ ID NO: 1 - DNA, 1228 bp, Homo sapiens
  CDS (35)...(1054)
taccggtccggaattcccgggtcgacgggggaagatggcgctacgtctgctgcggagggcggcgcgcggagctgcggcggcggcgctgctgaggctgaaagcgtctctagcagctgatatccccagacttggatatagttcctcatcccatcacaagtacatcccccggagggcagtgctttatgtacctggaaatgatgaaaagaaaataaagaagattccatccctgaatgtagattgtgcagtgctcgactgtgaggatggagtggctgcaaacaaaaagaatgaagctcgactgagaattgtaaaaactcttgaagacattgatctgggccctactgaaaaatgtgtgagagtcaactcagtttccagtggtctggcggaagaagacctagagacccttttgcaatcccggatccttccttccagcctgatgctaccaaaggtggaaagtcctgaagaaatccagtggtttgcagacaaattttcattccacttaaaaggccgaaaacttgaacaaccaatgaatttaatcccttttgtggaaactgcaatgggtttgctcaattttaaggcagtgtgtgaagaaaccctgaaggtcgggcctcaagtaggtctctttctagatgcagtcgtttttggaggagaagactttcgagccagcataggtgcaacaagtagtaaagaaaccctggatattctctacgcccggcaaaagattgttgtcatagcgaaagcctttggtctccaagccgtagatctggtgtacattgactttcgagatggagctgggctgcttagacagtcacgagaaggagccgccatgggcttcactggtaagcaggtgattcaccctaaccaaattgccgtggtccaggagcagttttctccttcccctgaaaaaattaagtgggctgaagaactgattgctgcctttaaagaacatcaacaattaggaaagggggcctttactttccaagggagtatgatcgacatgccattactgaagcaggcccagaacactgttacgcttgccacctccatcaaggaaaaatgatctgttaaatgaagctgtcatcaggctaaagggtattgaagctgcagagggatcaacttgtgcttgccagaggacgccaatgaagtttgaaacaccaacaatcagagattttgtttctgttcctcattaaatcatgagcttttgtgccgaaaaaaaaaaaaaaaaaaaaaa

SEQ ID NO: 2 - protein, 340 aa, Homo sapiens
MALRLLRRAARGAAAAALLRLKASLAADIPRLGYSSSSHHKYIPRRAVLYVPGNDEKKIKKIPSLNVDCAVLDCEDGVAANKKNEARLRIVKTLEDIDLGPTEKCVRVNSVSSGLAEEDLETLLQSRILPSSLMLPKVESPEEIQWFADKFSFHLKGRKLEQPMNLIPFVETAMGLLNFKAVCEETLKVGPQVGLFLDAVVFGGEDFRASIGATSSKETLDILYARQKIVVIAKAFGLQAVDLVYIDFRDGAGLLRQSREGAAMGFTGKQVIHPNQIAVVQEQFSPSPEKIKWAEELIAAFKEHQQLGKGAFTFQGSMIDMPLLKQAQNTVTLATSIKEK

SEQ ID NO: 3 - DNA, 1240 bp, Rattus norvegicus
  CDS (10)...(1023)
gtcgggaagatggcgctgtgtgtgctgcagaacgcggttctcggagcggcggcgctgcccaggctaaaggcatccctcgtggccagtgtctgcaggcctgggtacagctccttgtccaatcacaagtatgtccctcggagggcagtgctctatgtgcctgggaatgacgaaaagaaaataaggaagatcccatctttgaaagtagattgtgccgtgctggactgcgaggacggtgtcgcggaaaacaaaaagaacgaagctcgattgagaatagcaaaaactcttgaagactttgacctgggcacaacggaaaagtgtgtgagaatcaactcagtgtctagtggtctggctgaagccgacctggagacatttctacaggccagagttcttccttctagtctgatgctaccaaaggtagaaggtcctgaagaaatccagtggttttcagacaaattttcactccacttaaaaggccgaaagcttgaacaaccaatgaatttaatcccctttgtggagactgcaatgggtttgctcaattttaaggcagtgtgtgaagaaacactaaagattggacctcaagtgggtcttttcctagacgcagtcgtttttggaggagaagattttcgagccagcataggtgcaacaagcaacaaggacacccaagacatcctctatgcccgacagaagattgtcgtcacggcaaaggcctttggcctacaagccatagatcttgtatacatcgacttccgggatgaagatggacttctcagacagtctagagaagcagccgccatgggcttcactggtaaacaggtgatccaccctaaccagattgccgtggtacaggaacagtttactccaaccccggaaaaaattcggtgggctgaagagctgattgctgccttcaaagaacaccagcagttgggaaagggagcctttactttccaaggaagtatgatcgacatgccattactgaagcaggcccagaacattgttatgcttgccacatccatcaaggaaaaatgatctgttaaatgaagcccttctcaggcgggctgaatggataccgtgggtttcttaaagacaaaccacagtacccagggcagcttgctagcacgccggatcgggacctcatctcacgttagtcacaacgccccctccccttgcatcttactgacccgatattctaaaaataaacgttcttcccttggaatctaaaaaaaaaaaaaaaaaaaaaaaa

SEQ ID NO: 4 - protein, 338 aa, Rattus norvegicus
MALCVLQNAVLGAAALPRLKASLVASVCRPGYSSLSNHKYVPRRAVLYVPGNDEKKIRKIPSLKVDCAVLDCEDGVAENKKNEARLRIAKTLEDFDLGTTEKCVRINSVSSGLAEADLETFLQARVLPSSLMLPKVEGPEEIQWFSDKFSLHLKGRKLEQPMNLIPFVETAMGLLNFKAVCEETLKIGPQVGLFLDAVVFGGEDFRASIGATSNKDTQDILYARQKIVVTAKAFGLQAIDLVYIDFRDEDGLLRQSREAAAMGFTGKQVIHPNQIAVVQEQFTPTPEKIRWAEELIAAFKEHQQLGKGAFTFQGSMIDMPLLKQAQNIVMLATSIKEK

SEQ ID NO: 5 - DNA, 1286 bp, Mus musculus
  CDS (53)...(1066)
ctaatacgactcactatagggcaagcagtggtggcctactggagtcgggaagatggcgttgtgcgtgctgcggaacacggttcgtggagcggcggcgctgcccaggctgaaggcatcccacgtggtcagtgtctacaagcctagatacagctccttgtccaatcacaagtatgtcccccggagagcagtgctctatgtccctgggaatgacgaaaagaaaataaggaagatcccgtccttgaaagtagattgtgccgtgctggactgcgaggatggtgttgcagaaaacaaaaagaatgaagctcgattgagaatagcaaaaactcttgaagactttgacctgggcacaacggaaaagtgtgtgagaatcaactcggtgtctagtggtctggctgaagtcgacctggagacatttctacaggccagagttcttccctctagtctgatgctaccaaaggtagaaggtcctgaagaaatccgatggttttcagacaaattttcactccacttaaaaggccgaaaacttgaacaaccaatgaatttaatcccctttgtggagactgcaatgggtttgctcaattttaaggcagtgtgtgaagaaacactaaagactggacctcaagtgggtctttgcctagatgcagtcgtttttggaggagaagattttcgagccagcataggtgcaacaagcaacaaagacacccaagacatcctctatgcccggcagaaggttgttgtcacggcaaaagcctttggcctacaagccatagatcttgtgtacattgacttccgagatgaagacggacttctcagacagtcaagggaagcagccgccatgggcttcactggtaaacaggtgatccaccctaaccagatcgcagtggtacaggaacagtttactccaaccccagaaaaaattcagtgggctgaagagctgattgctgccttcaaagaacaccagcagctgggaaagggagcctttactttccgaggaagtatgatcgacatgccattactgaagcaggcccagaacattgttacgcttgccacatccatcaaggaaaaatgatctgttaaatgaagccctcctcaggcgggctgaatggatactgtgggcttcttaaagacaaaccgcagtacccagggcagcttgctagcatgctggatcaggacctcatttcacgttagtcacaacgccccctccccttgtatcttactgacccgctatgctaaaaataaactttcttcctctggagtctaaaaaaaaaaaaaaaaaaaaaaaaaaa

SEQ ID NO: 6 - protein, 338 aa, Mus musculus
MALCVLRNTVRGAAALPRLKASHVVSVYKPRYSSLSNHKYVPRRAVLYVPGNDEKKIRKIPSLKVDCAVLDCEDGVAENKKNEARLRIAKTLEDFDLGTTEKCVRINSVSSGLAEVDLETFLQARVLPSSLMLPKVEGPEEIRWFSDKFSLHLKGRKLEQPMNLIPFVETAMGLLNFKAVCEETLKTGPQVGLCLDAVVFGGEDFRASIGATSNKDTQDILYARQKVVVTAKAFGLQAIDLVYIDFRDEDGLLRQSREAAAMGFTGKQVIHPNQIAVVQEQFTPTPEKIQWAEELIAAFKEHQQLGKGAFTFRGSMIDMPLLKQAQNIVTLATSIKEK

SEQ ID NO: 7 - DNA, 25 bp, Artificial Sequence
  Primer
aagctgccctgggtactgtggtttg

SEQ ID NO: 8 - DNA, 25 bp, Artificial Sequence
  Primer
cagattgccgtggtacaggaacagt

SEQ ID NO: 9 - DNA, 19 bp, Artificial Sequence
  Forward primer
cacaggaaacagctatgac

SEQ ID NO: 10 - DNA, 19 bp, Artificial Sequence
  Reverse primer
ccagtcacgacgttgtaaa

SEQ ID NO: 11 - DNA, 23 bp, Artificial Sequence
  3′ RACE primer
ttgtgcgtgctgcggaacacggt

SEQ ID NO: 12 - DNA, 24 bp, Artificial Sequence
  5′ RACE primer
gcagtccacacggcacaatctact

SEQ ID NO: 13 - DNA, 25 bp, Artificial Sequence
  Forward PCR primer
tgggccctactgaaaaatgtgtgag

SEQ ID NO: 14 - DNA, 20 bp, Artificial Sequence
  Reverse PCR primer
ggcggctccttctcgtgact